(19) United States
(12) Patent Application Publication
(10) Pub. No.: US 2004/0166069 A1
(43) Pub. Date: Aug. 26, 2004

(54) Boosting Tyrosinase Inhibiting Activity of Skin Whitening and Sunscreen Compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/248,817

(22) Filed: Feb. 21, 2003

(51) Int. Cl. A61K 7/42; A61K 7/135

(52) U.S. Cl. 424/59; 424/62

(57) ABSTRACT

The present invention relates to boosting of the skin whitening benefits of commonly used tyrosinase inhibitors, tyrosinase competitors, and melanin and other color bodies' reducing agents. The activity of such compositions can be synergistically enhanced by the inclusion of at least one antioxidant composition. This synergistic benefit is further increased if a UV-inhibitor or UV-absorber is also included in such compositions. This is both unexpected and surprising because most such tyrosinase inhibitors, tyrosinase competitors, or melanin reducing agents are also known to possess antioxidant and UV-absorbing properties. Thus, the skin whitening benefits are synergistically increased by combining at least one of the following; (i) a tyrosinase inhibiting agent, or (ii) a tyrosinase competing agent, or (iii) a melanin reducing agent, or (iv) combinations thereof; with at least one antioxidant composition. The additional inclusion of at least one UV inhibitor or absorber composition provides further enhancement of skin whitening effects from such tyrosinase inhibiting, tyrosinase competing or melanin reducing agents that are formulated in combination with an antioxidant composition. In a related application, the skin whitening property of a sunscreen composition can be boosted by the inclusion of an antioxidant composition and a tyrosinase inhibitor, tyrosinase competitor, or melanin and other color bodies reducing composition.

### BACKGROUND OF INVENTION

The cosmetic treatment of skin to produce a visible even-tone has been practiced since ancient times. The use of plant-derived extracts and salves to whiten or brighten dark colored skin has been very popular among Asian, African, and South American cultures. The even toning of age-related dark spots, skin pigmentation, freckles, and other skin pigmentation disorders with skin lightening products is gaining popularity among people of light-colored skin as well.

Hydroquinone is one of the ingredients of choice, mostly because of its status as a FDA approved OTC drug active ingredient for skin whitening compositions. Kojic acid and arbutin, which are chemically related to hydroquinone, are also commonly used.

Topical applications of ascorbic acid and its esters are also claimed to have skin-lightening property. Several botanical-based ingredients with claims such as “helps reduce the appearance of minor skin discoloration”, “helps brighten skin and even-out skin tone”, “helps reduce the appearance of dark spots, age-related spots, and freckles”, and modifications of the above claims, have been disclosed.

The color of human skin is differentiated by the nature and quantity of natural pigment, melanin, present in the epidermal layers of skin. The formation of melanin from amino acid tyrosine involves several biogenetic steps mediated initially by enzyme tyrosinase. Tyrosine is first oxidized by tyrosinase to dihydroxyphenylalanine (I-dopa), then to dopaquinone. Dopaquinone is then converted into eumelanin (black, white, and Asian skin types; skin color dependent on the quantity of eumelanin in skin), or phaeomelanin (red-haired skin types). This conversion proceeds through the intermediate formation of leucodopachrome and dopachrome (eumelanin), or cysteinyldopa (phaeomelanin).

The successful development of a skin whitening formulation can incorporate two plausible approaches to skin depigmentation based on the mechanism of melanin formation discussed above; (1) The inhibition of melanin biosynthesis, and (2) conversion of melanin and its colored precursors (dopachrome, leucochrome) into colorless entities by reducing agents. The inhibition of melanin biosynthesis itself may be achieved by three mechanisms (1) The inactivation of enzyme tyrosinase, or (2) The competitive replacement of tyrosinase substrates, tyrosine or I-dopa with other chemically related compositions, and (3) The inhibition of the oxidation/hydroxylation of tyrosine to produce I-dopa without inhibiting tyrosinase.

On the practical grounds, four rational approaches to skin whitening compositions include (1) the inhibition of tyrosinase, (2) the competitive replacement of the substrates for tyrosinase, (3) The inhibition of the oxidation/hydroxylation of tyrosine to form I-dopa, and (4) the conversion of colored biochemical species (such as melanin, dopachrome, leucochrome, eumelanin, or phaeomelanin) into colorless or less colored materials. It has been generally known to utilize a tyrosinase inhibitor or a tyrosine competitor (to block enzyme tyrosinase), and a reducing agent (to convert melanin and its pigmented precursors into colorless, or less colored biochemical entities) for skin whitening compositions.

A great number of skin whitening compositions have become commercially available. Most of those preparations are tyrosinase inhibitors. For example, Gattefosse markets “Gatulin Whitening”, which is a mixture of Aspergillus Orizae and Licorice extracts. Gattefosse also markets “Synerlight”, which is a mixture of Sophora root extract, kiwi water, and ascorbic acid, and “Mulberry Extract”, for skin whitening applications. “Etioline”, which is a mixture of Mitracarpus scaber extract and bearberry (Arctostaphylus uva ursi) extract, is skin whitening tyrosinase inhibitor marketed by Sederma. “Melaslow” and “Melaclear” are two additional skin-whitening compositions marketed by Sederma, both of which are based on tyrosinase inhibitors. “Dermalight” is a tyrosinase inhibiting composition based on nasturtium petals marketed by Silab. “Clariskin”, also offered by Silab, is another tyrosinase inhibitor derived from wheat germ extract. “Tyrostat-09” and “Tyrostat-11” marketed by Fytokem are both based on tyrosinase inhibitors obtained from a Canadian plant, and further disclosed in U.S. Pat. No. 6,521,267 (Steck). Alpaflor offers “Gigawhite”, a skin whitening composition based on a mixture of several tyrosinase inhibiting botanical extracts including Malva sylvestris, Mentha piperita, Primula veris, Alchemilla vulgaris, Veronica officinalis, Melissa officinalis, and Achillea millefolium.

A much smaller number of compositions are available that act by reducing mechanism. The examples include hydroquinone, arbutin, and ascorbic acid and its derivatives.

The author of the present invention has published an article on skin whitening agents (S.Gupta, “Plant-based Skin Whitening Cosmetics”, Household and Personal Products Industry (HAPPI), page 90, April 2001). Some of the important compositions include Paper Mulberry (Broussonetia kazinoke); the extracts of root and bark are potent tyrosinase inhibitors. Mitracarpe (Mitracarpus scaber), the leaf extract of this tropical plant contains harounoside, a hydroquinone derivative with strong anti-tyrosinase activity. Also, a mixture of this extract with bearberry has shown potent tyrosinase inhibiting and skin whitening properties. Bearberry (Arctostaphylos uva ursi), the leaf extract of this plant contains hydroquinone derivatives, arbutin and methyl arbutin with skin whitening attributes. Yellow Dock (Rumex crispus, Rumex occidentalis), this extract has shown excellent anti-tyrosinase and skin whitening attributes. The chemical constituents of this recently discovered material responsible for skin whitening activity are unknown. Glutathione: Glutathione has been used in skin lightening compositions. Reduced form of glutathione has a dual role in the depigmentation of colored skin. The mechanism of action may involve competitive binding with the color forming precursors of tyrosine (dopaquinone) to form less-colored phaeomelanin. It may also act as a reducing agent to effect the bleaching of the colored melanin precursors. Leukocyte Extract, it is a fractionated blend of biotechnology-derived peptides with tyrosinase inhibiting activity. The possible mode of action may involve its ability to denature protein backbone of enzyme tyrosinase, thus inhibiting that enzyme. Aspergillus orizae, this fermentation-derived material contains kojic and lactic acids. Kojic acid is a known tyrosinase-inhibiting, skin color-reducing ingredient. Licorice Root (Glycyrrhiza glabra), this botanical has been used for a variety of skin disorders since ancient history. Recent studies have shown its promising skin whitening activity. Hispaglabridin, glabridin, isoliquiritin, and their derivatives present in this botanical have striking structural similarity to other dihydroxybenzene-type skin whitening compounds. Rosmarinic Acid, Tetrahydrocurcumin, and Green Tea Extract; these all possess antityrosinase activity and skin lightening properties. Yohimbe (Pausinystalia yohimbe), the extract of yohimbe bark contains alkaloid Yohimbine and its isomers. It is reported to inhibit melanin biosynthesis, hence its application in cosmetic skin bleaching formulations. Cang Xu, Bai Xu: These Chinese folk medicines have been used for centuries for skin whitening, age spot removal, and skin tone enhancement applications. These are known to possess tyrosinase-inhibiting effects. The rhizomes of Cang Xu (Atractylodes lancea) and related plant, Bai Xu (Atractylodes macrocephalia) have been used for at least 2000 years in China for the removal of dark spots on the face and hand and skin whitening liniments. Atractylodin, an acetylenic furan derivative present in high amounts in these extracts may be a tyrosinase inhibitor. Bai Xian Pi (Dictamnus albus) preparations from root bark have antifungal and skin whitening attributes. Hu Zhang (Polygonum cuspidatum) has been used in China with a recorded history of over 2000 years. It contains anthraquinone derivative, emodin and stilbene derivative, resveratrol, which have recently been shown to possess tyrosinase-inhibiting activity. Gao Ben (Ligusticum sinensis) and its close relative, Chuanxiong (Ligusticum chuanxiong) have been used for dark spots, freckles, acne rosacea, and skin whitening applications dating back to 800 B.C. Ferulic acid present in these extracts may be a tyrosinase inhibitor. Fangfeng (Saposhnikovia divaricata) has been used for dark spots removal and skin whitening preparations.

It has also been known that UV and sunlight stimulate the production of melanin as a result of body's own protective response to such external stimuli. The use of sunscreen compositions has been practiced to reduce such skin darkening effects of UV and sunlight.

Relative to prior art knowledge of skin whitening compositions, several examples can be cited. To date, the best-known active substance for de-pigmentation is hydroquinone, a bleaching agent. Hydroquinone, however, does not inhibit melanin biosynthesis: it bleaches existing melanin. If applied over long periods of time, hydroquinone can have serious side effects, which has led to its being permitted only in limited concentrations in some countries, and to its being completely forbidden for applications in cosmetic products in other countries. Furthermore, hydroquinone leads to permanent de-pigmentation, and thus to increased photosensitivity of the skin when exposed to UV light. Better-tolerated skin lightening substances currently being used are of natural origin, e.g., arbutin (from the leaves of the common bearberry, Uva ursi), liquorice extract (from liquorice root), ascorbic acid (vitamin C from citrus fruits) and their derivatives, as well as kojic acid (from carbohydrate solutions under the effect of certain bacteria). These substances, which are highly soluble in water, act on the tyrosinase as competitive inhibitors; however, they are unstable in some formulations, and have the disadvantage that only very small quantities penetrate the deeper skin layers and reach the melanocytes in the basal membrane. A further disadvantage of these substances is their low level of efficacy, which necessitates their being used in high concentrations. Compared to the quantity of hydroquinone used, 17 times as much ascorbic acid and over 100 times as much arbutin is required to achieve a similar effect.

U.S. patent application Ser. No. 20030017997 (Yokota et al.) discloses certain melanogenesis inhibitor dihydroxyphenyl compounds for skin whitening.

U.S. patent application Ser. No. 20020176903 (Kuno et al.) discloses olive extracts for skin whitening benefits. These appear to be tyrosinase inhibitors.

U.S. patent application Ser. No. 20020141956 and 20020071816 (Perricone) disclose hydroxytetronic acid and its derivatives as anti-tyrosinase skin whitening compositions.

U.S. patent application Ser. No. 20010029253 (Park et al.) discloses certain 1,3-beta-glucan derivatives for skin whitening applications.

U.S. Pat. No. 6,365,137 (Aust et al.) discloses certain polyhydroxycarboxylic acids an anti-tyrosinase skin whitening agents.

U.S. Pat. No. 6,083,976 (Padmapriya et al.) discloses certain aloesin derivatives as tyrosinase inhibiting skin whitening agents.

U.S. Pat. No. 5,993,835 (Mishima) discloses certain melanin complexing agents for skin whitening applications.

U.S. Pat. No. 5,980,904 (Leverett et al.) discloses a skin whitening composition that contains bearberry extract and a reducing agent to boost the skin-whitening efficacy of beaberry extract. The reducing agents of preferred compositions were all formaldehyde donors. The formaldehyde donors are forbidden in many countries of the world due to their toxicity and skin sensitization problems.

U.S. Pat. No. 5,916,915 (Hong et al.) discloses certain ascorbic acid derivatives for skin whitening applications.

U.S. Pat. No. 5,824,327 (Whittemore et al.) discloses certain derivatives of kojic acid, suitable as tyrosinase inhibiting skin-whitening agents. These derivatives circumvent the problems of other kojic acid derivatives, for example Nagai, et al, U.S. Pat. No. 4,278,656 forms a skin whitener cosmetic composition from a kojic acid ester with an aliphatic carboxylic acid. The composition utilizes water and the non-dipalmitate esters used will turn color. The U.S. Pat. No. 4,369,174 to Nagai, et al, discloses a skin whitener cosmetic composition utilizing a kojic acid ester as an active ingredient. The composition utilizes water and will turn color. In the U.S. Pat. No. 4,696,813 to Higa, the skin whitener cosmetic composition comprises placenta and kojic acid. It does not use esters, does utilize water and will turn color. Hatae, et al, U.S. Pat. No. 4,847,074 relates to a kojic acid containing whitener cosmetic composition that includes cyclodextrins for improved stability, i.e. to compensate for color changes. U.S. Pat. No. 4,919,921 (Hatai) the cosmetic composition comprises kojic acid or an ester of kojic acid and Vitamin C, but this composition will change color in formulation. Hara, U.S. Pat. No. 4,948,577 relates to a skin whitener composition comprised of kojic acid or derivatives thereof with 4-(1,1-dimethylethyl)-4.sup.1 methoxydiben-zoylmethan formulated therein. The composition will turn color, and prevents normal tanning but is not true skin bleach. The skin whitener composition of U.S. Pat. No. 4,985,255 (Higa) comprises placental extract of pregnant cows and kojic acid or a derivative thereof. The mixture uses water and will turn color. U.S. Pat. No. 4,985,455 (Motono) incorporates an ultraviolet absorber, B cyclodextrin and ethylendiaminetetracetic acid to eliminate or reduce discoloration in a kojic acid or derivatives thereof skin whitener cosmetic composition. U.S. Pat. No. 4,990,330 (Oyama) an amino compound is included in a kojic acid based skin whitener cosmetic composition to inhibit melanin synthesis.

The skin lightening composition of Meybeck, et al, (U.S. Pat. No. 5,164,182) is a composition containing a mulberry extract incorporated into hydrated lipidic lamellar phases of liposomes. In the whitener composition of Maybeck, U.S. Pat. No. 5,279,834 hydroquinone and/or kojic acid or a derivative thereof is partially incorporated into liposomes.

U.S. Pat. No. 5,427,775 (Sakai) the whitening composition comprises teprenone and one or more substances selected from the groups consisting of kojic acid, L-ascorbic acid and arbutin.

U.S. Pat. No. 5,609,875 (Hadas) discloses licorice extract compositions that possess boosted skin whitening efficacy from the inclusion of certain hydroxy acids. Since hydroxy acids themselves are known for their skin whitening property, such a disclosure is not surprising or unexpected.

U.S. Pat. No. 6,214,352 (Matsukawa) discloses extracts of Gardenia, Sophora and Rosa species of plants to possess tyrosinase-inhibiting activity.

U.S. Pat. No. 6,165,982 (Yamada) discloses compositions with sericin to be tyrosinase inhibitors for skin whitening application.

U.S. Pat. No. 5,773,014 (Perrier et al.) discloses tyrosinase-inhibiting properties of extracts of mulberry, saxifrage, grape and scutellaria root.

The above references again illustrate that tyrosinase inhibitors represent the greatest number of skin whitening compositions. It would thus be advantageous if synergistic combinations could be devised that can boost the skin whitening efficacy of such tyrosinase inhibitors, thus requiring their use in lower amounts in skin whitening compositions.

On the basis of foregoing discussion, the following five factors become evident that can control skin depigmentation: (1) Inhibition of melanin biosynthesis; and (2) Conversion of melanin or melanin precursors to decolorized forms; and (3) Inhibition of tyrosinase enzyme; and (4) Competitive replacement of tyrosinase substrates; and (5) Inhibition of UV. These five factors can be interrelated to each other.

Surprisingly, it has now been discovered that the skin whitening benefits of commonly used tyrosinase inhibitors, tyrosinase competitors, and melanin reducing agents can be synergistically enhanced by the inclusion of an antioxidant composition. This synergistic benefit is further increased if a UV inhibitor is also included in such compositions. Thus, the skin whitening benefits are synergistically increased by combining at least one (i) a tyrosinase inhibiting agent, or (ii) a tyrosinase competing agent, or (iii) a melanin reducing agent, or (iv) combinations thereof, with at least one antioxidant composition. The additional inclusion of at least one UV inhibitor agent provides further enhancement of skin whitening effects from such tyrosinase inhibiting, or tyrosinase competing, or melanin reducing agents.

### SUMMARY OF INVENTION

I have now discovered that that the skin whitening benefits of commonly used tyrosinase inhibitors, tyrosinase competitors, or melanin reducing agents can be synergistically enhanced by the inclusion of at least one antioxidant composition. This synergistic benefit is further increased if at least one UV inhibitor or absorber is also included in such compositions. This is both unexpected and surprising because most tyrosinase inhibitors, tyrosinase competitors, or melanin reducing agents are also known to possess antioxidant and UV-absorbing properties. Thus, the skin whitening benefits are synergistically increased by combining at least one of the following; (i) a tyrosinase inhibiting agent, or (ii) a tyrosinase competing agent, or (iii) a melanin reducing agent, or (iv) a combinations thereof; with at least one antioxidant composition. The additional inclusion of at least one UV inhibitor or absorber composition provides further enhancement of skin whitening effects from such tyrosinase inhibiting, tyrosinase competing or melanin reducing agents.

### DETAILED DESCRIPTION

A great number of skin whitening compositions have become commercially available. Most of those preparations are tyrosinase inhibitors, as noted in the Background section of this invention. It would thus be highly desirable to further enhance the skin whitening power of such ingredients or compositions without utilizing formaldehyde generating reducing agents or acids with low pH. It would be further desirable to synergistically enhance such skin whitening power and also provide other skin beneficial attributes, such as anti-aging benefits, anti-wrinkle benefits, skin smoothing and skin pliability enhancement, and collagen synthesis activation.

I have now discovered that tyrosinase enzyme inhibiting property of various compositions can be synergistically boosted by the inclusion of at least one antioxidant composition. In addition to enhanced skin whitening from boosted anti-tyrosinase effects, such compositions can also provide anti-aging, anti-wrinkle, anti-acne, anti-rosacea, and skin smoothing benefits. This is both surprising and unexpected, since most tyrosinase inhibitors themselves possess antioxidant properties. The inclusion of an antioxidant in melanin reducing compositions, such as hydroquinone and arbutin, also boosts the skin whitening effects of such reducing agent compositions. The skin whitening boosting effect is further enhanced if a UV absorber is also included in such combinations of tyrosinase inhibitor and antioxidant. Thus, the skin whitening benefits are synergistically increased by combining at least one of the following; (i) a tyrosinase inhibiting agent, or (ii) a tyrosinase competing agent, or (iii) a melanin reducing agent, or (iv) a combination thereof; with at least one antioxidant composition. The additional inclusion of at least one UV inhibitor or absorber composition provides further enhancement of skin whitening effects from such tyrosinase inhibiting, tyrosinase competing, or melanin reducing agents.

Although not bound by any theory, it is my belief that the mechanism of antioxidant composition to synergistically enhance anti-tyrosinase activity involves the blocking of the oxidation/hydroxylation of tyrosine substrate. It is not unreasonable to propose that an antioxidant can stop the oxidation step. It may thus be the first known example of a composition that works by preventing the oxidation/hydroxylation of tyrosine, and not by blocking tyrosinase enzyme itself or acting as a competitive substrate for tyrosinase.

Relative to the selection of tyrosinase inhibitors, tyrosinase competitors, and melanin and other color body reducing agents, any such compositions can be selected in any proportions or combinations. As is illustrated in the Examples section, even currently commercially available skin whitening compositions can be further improved in their skin whitening property by the present invention. Thus, any of the compositions referenced in this invention, and any future tyrosinase inhibitors, tyrosinase competitors, and melanin and other color body's reducing agents can be boosted in their skin whitening performance by the present invention.

Relative to the nature of antioxidants that can be used in the present invention, there appears to be no limitation observed, as both water-soluble and oil-soluble antioxidants are equally effective. This is highly advantageous, since the antioxidant of choice can be selected on the basis of the water or oil solubility desired for a specific composition. For water-in-oil or oil-in-water emulsions, the inclusion of both a water-soluble and an oil-soluble antioxidant would be beneficial. The weight ratio of such antioxidants is not critical, since most antioxidants have their maximum efficacy in a wide range of percentages in topical compositions. For starting purposes, a 1:1 ration of water-soluble and oil-soluble antioxidants is recommended.

In an article written by the author of the present invention (S. Gupta, Antioxidants. Formulation of Cosmetic Delivery Systems, Household and Personal Products Industry (HAPPI), 56 (July 2001), it has been illustrated that antioxidants can belong to a variety of chemical classes, such as polyphenols, carotenoids, flavones, and such. It is advantageous if such antioxidants are combined in such a manner that compositions of different groups are combined rather than several compositions of the same chemical group.

Relative to the nature of antioxidant compositions, the selection can be made from including, but not limited to, Ascorbic acid, Ascorbic acid Esters, Ascorbic acid glucosides, Ascorbic acid salts and other derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Pycnogenol, Grape Seed Extract, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), pyridoxine, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, Ecklonia cava extract, Spondias mombin extract, Maprounea guianensis extract, Walteria indica extract, Gouania blanchetiana extract, Cordia schomburgkii extract, Randia armata extract, Hibiscus furcellatus extract, and combinations thereof.

The use of a UV absorber to further enhance the synergistic skin whitening property of tyrosinase inhibitor/antioxidant combination of the present invention is both unexpected and surprising. However, it does open up another new, yet unexplored market of developing sunscreen agents that also have tyrosinase-inhibiting skin whitening effect. The UV absorber to antioxidant ratio is not critical. However, for starting compositions, a 1:1 ration of UV absorber to antioxidant is recommended.

UV inhibitor or UV absorber can be selected from including, but not limited to Kaempferia galanga extract, aminobenzoic acid, Cinoxate, Ethylhexyl methoxycinnamate, Avobenzone, Homosalate, Lawsone, Menthyl anthranilate, Octocrylene, Ethylhexyl salicylate, oxybenzone, Padimate-O, Benzophenone-3, Benzophenone-4, Sulisobenzone, Titanium dioxide, Zinc oxide, Trolamine salicylate, Glyceryl aminobenzoate, and combinations thereof.

In a similar field, the present invention can also synergistically add surprising skin whitening and skin lightening attributes to a sunscreen composition. Thus, a sunscreen composition with at least one tyrosinase inhibitor and at least one antioxidant can provide UV protection as well as synergistic skin whitening and skin lightening effects. This is of special significance, since exposure to sunlight is well known to cause the darkening of skin due to melanocyte activation. This can be achieved by the simple addition of a tyrosinase inhibitor and an antioxidant composition to any sunscreen product of any sun protection factor (SPF) value. As noted in the Examples section of the present invention, even a commercially marketed sunscreen composition can be boosted for its skin whitening power.

For the determination of skin whitening and skin lightening effect, a new methodology has been developed in the present invention. Human volunteers were selected with both light and medium dark skin types. An area on the forearm was marked with 1-inch square boxes. The products under test and controls were applied to these areas on an equal weight basis. These areas were then exposed to artificial solar lamp equipment (solar simulator equipment) that is very similar to that used for the SPF determination of sunscreen products. These areas were exposed to artificial sunlight for a period of time that only produced skin-darkening effect on an untreated area without causing any erythema. The treated areas were then exposed to artificial solar light for the same amount of time. The skin lightening effect was then determined both visually and by a light meter reading. In all cases, the skin areas that showed lesser amount of darkening than the corresponding controls were considered as positive response results. In all cases illustrated in the Examples section, the compositions made according to claims section of the present invention showed lighter colored skin (less darkening) than the corresponding control samples that did not contain such compositions. The skin lightening effect was observed on a scale of 1 to 5 (5 means highest amount of skin lightening effect), the differences were at least one scale unit (i.e. observable with naked eye). The following order of skin whitening was noted: antioxidant+UV absorber>antioxidant>no antioxidant/yes UV absorber>no antioxidant/no UV absorber.

### EXAMPLES

The following examples are presented to illustrate presently preferred practice thereof. These examples illustrate how the efficacy of commercially available tyrosinase inhibiting compositions can be boosted with the inclusion of at least one antioxidant composition. These examples also include the formulation of consumer desirable lotion, cream, and other such compositions for their retail marketing. As illustrations they are not intended to limit the scope of the invention. All quantities are in weight %.

### Example 1

Skin Lightener Serum. This product is based on a combination of skin color reducing properties of ascorbic acid and glutathione, tyrosinase inhibiting property of paper mulberry extract, and skin whitening properties of Arctostaphylos uva ursi and Mitracarpus scaber. Ingredients % Weight (1) Deionized water 10.0 (2) Ascorbic acid 5.0 (3) Methylpropanediol 69.0 (4) Dimethicone copolyol 4.0 (5) Arctostaphylos uva ursi Leaf extract (and) Mitracarpus Scaber extract 1.0 (6) Broussonetia kazinoke (Paper mulberry) Root Extract 0.5 (7) Glutathione (reduced) 0.5 (8) Preservatives qs (9) Acrylates/aminoacrylates/ C10-30 Alkyl PEG-20 Itaconate Copolymer 10.0 Procedure. Make main batch by mixing 3 to 8 at room temperature. Pre-mix 1 and 2 to a solution and add to main batch with mixing. Gradually add 9 till desired viscosity is reached. The product has a clear to slightly hazy syrup-like appearance (pH 3.5 4.0), typical of a skin serum product. It is absorbed rapidly with a silky smooth skin feel. This formula is adaptable for other botanical ingredients also.

### Example 2

Skin Lightener Cream. This product is based on a combination of skin whitening properties of Arctostaphylos uva ursi and Mitracarpus scaber, tyrosinase inhibiting property of Rumex crispus extract, and skin color reducing properties of sodium metabisulfite. Ingredients % Weight (1) Deionized water 79.0 (2) Cetearyl alcohol (and) dicetyl phosphate (and) Ceteth-10 phosphate5.0 (3) Cetyl alcohol 2.0 (4) Glyceryl stearate (and) PEG-100 stearate4.0 (5) Caprylic/capric triglyceride5.0 (6) Arctostaphylos uva ursi Leaf extract (and) Mitracarpus Scaber extract 3.0 (7) Rumex crispus extract 1.0 (8) Sodium metabisulfite 0.5 (9) Niacinamide ascorbate 0.5 (10) Preservatives qs (11) Sodium hydroxide (for pH adjustment) qs. Procedure. Mix 1 to 5 and heat to 75-80° C. Adjust pH to 4.0 4.5. Cool to 35-40° C. with mixing. Add 6 to 11 with mixing. Adjust pH to 4.0-4.5, if necessary. White to off-white cream.

### Example 3

Skin Depigmentation Facial Mask Composition Ingredient % (1) Chitosan 5.0 (2) Lactic Acid 5.0 (3) Glycerin 17.7 (4) Water 70.6 (5) Yohimbine HCl 0.5 (6) Niacinamide Lipoate 0.5 (7) Glutathione0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 4

Boosting Anti-tyrosinase activity of “Gigawhite” Composition with an Antioxidant. Ingredient % (1) “Gigawhite” (obtained from Alpaflor Company, Switzerland) 90.0 (2) PEG-6 9.5 (3) Resveratrol 0.5 Procedure. Mix 2 and 3 to a solution. Add to 1 and mix. A thin, light brown liquid is obtained.

### Example 5

Boosting Tyrosinase inhibiting property of “Tyrostat-09” Composition with an Antioxidant. Tyrostat-09 composition was obtained from Fytokem, Saskatchewan, Canada. Ingredient % (1) Niacinamide ascorbate 5.0 (2) Deionized water 10.0 (3) Tyrostat-09 85.0. Procedure. Mix (1) and (2) to a solution. Add this solution to (3) and mix. A light amber liquid is obtained.

### Example 6

Boosting Tyrosinase inhibition of “Etioline” composition with an Antioxidant. “Etioline” was obtained from Sederma/Croda, USA. Ingredient % (1) Niacinamide mandelate 5.0 (2) Deionized water 10.0 (3) Etioline 85.0 Procedure. Mix (1) and (2) to a solution. Add to (3) and mix. An amber solution was obtained.

### Example 7

Boosting Anti-Tyrosinase action of “Gatuline Whitening” composition with an Antioxidant. “Gatulin Whitening” was obtained from Gattefosse Corporation.

Ingredient % (1) Yohimbine HCl 2.5 (2) Deionized water 7.5 (3) Gatulin Whitening 90.0 Procedure. Mix (1) and (2) to a solution. Add this to (3) and mix. A light amber solution was obtained.

### Example 8

Boosting Tyrosinase Inhibitory activity of “Dermalight” Composition with an Antioxidant. “Dermalight” was obtained from Silab, France. Ingredient % (1) Glutathione ascorbate 1.5 (2) Deionized water 5.0 (3) “Dermalight” 93.5 Procedure. Mix (1) and (2) to a solution. Add this to (3) and mix. An amber solution was obtained.

### Example 9

Boosting Anti-tyrosinase activity of “Gigawhite” Composition with Antioxidant and UV Absorber Combination.

Ingredient % (1) “Gigawhite” (obtained from Alpaflor Company, Switzerland) 89.5 (2) PEG-6 9.5 (3) Resveratrol 0.5 (4) Ethylhexyl Methoxycinnamate 0.5 Procedure. Mix (2), (3) and (4) to a solution. Add to (1) and mix. A thin, light brown liquid is obtained.

### Example 10

Boosting Tyrosinase inhibiting property of “Tyrostat-09” Composition with Antioxidant and UV Absorber Combination Composition. Tyrostat-09 composition was obtained from Fytokem, Saskatchewan, Canada. Ingredient % (1) Niacinamide ascorbate 5.0 (2) Benzophenone-4 0.5 (3) Deionized water 10.0 (4) Tyrostat-09 84.5. Procedure. Mix (1), (2) and (3) to a solution. Add this solution to (4) and mix. A light amber liquid is obtained.

### Example 11

Boosting Tyrosinase inhibition of “Etioline” Composition with Antioxidant and UV Absorber Combination . “Etioline” was obtained from Sederma/Croda, USA. Ingredient % (1) Niacinamide mandelate 5.0 (2) Benzophenone-0.5 (3) Deionized water 10.0 (4) Etioline 84.5 Procedure. Mix (1), (2) and (3) to a solution. Add to (4) and mix. An amber solution was obtained.

### Example 12

Boosting Anti-Tyrosinase action of “Gatuline Whitening” with Antioxidant and UV Absorber Combination. “Gatulin Whitening” was obtained from Gattefosse Corporation. Ingredient % (1) Yohimbine HCl 2.5 (2) Benzophenone-4 0.5 (3) Deionized water 7.5 (4) Gatulin Whitening 89.5 Procedure. Mix (1), (2) and (3) to a solution. Add this to (4) and mix. A light amber solution was obtained.

### Example 13

Boosting Tyrosinase Inhibitory activity of “Dermalight” with Antioxidant and UV Absorber Combination. “Dermalight” was obtained from Silab, France. Ingredient % (1) Glutathione ascorbate 1.5 (2) Benzophenone-4 0.5 (3) Deionized water 5.0 (4) “Dermalight” 93.0 Procedure. Mix (1), (2) and (3) to a solution.

Add this to (4) and mix. An amber solution was obtained.

### Example 14

A Sunscreen Composition with Boosted Skin Whitening Effect from Synergistic Tyrosinase Inhibition. Composition % (1) Niacinamide ascorbate 3.0 (2) Etioline 2.0 (3) A commercially marketed sunscreen composition with SPF-25 95.0 Procedure. Add (1) and (2) to a standard SPF 25 lotion. Mix. The SPF 25 lotion is thus transformed into a SPF 25 skin lightening sunscreen composition. The SPF value is not reduced, as niacinamide ascorbate also provides synergistic additional SPF value.

I claim:

1. A topical skin clarifying and skin whitening composition comprising:
(i) At least one (a) a tyrosinase inhibiting agent, or (b) a tyrosinase competing agent, or (c) a melanin reducing agent, or (d) combinations thereof, and
(ii) At least one antioxidant composition, and
(iii) A cosmetically or pharmaceutically suitable carrier composition.

2. A topical skin clarifying and skin whitening composition comprising:
(i) At least one (a) a tyrosinase inhibiting agent, or (b) a tyrosinase competing agent, or (c) a melanin reducing agent, or (d) combinations thereof, and
(ii) At least one antioxidant composition, and
(iii) At least one UV inhibitor composition, and
(iv) A cosmetically or pharmaceutically suitable carrier composition.

3. A topical sunscreen composition with boosted tyrosinase inhibition comprising:
(i) At least one (a) a tyrosinase inhibiting agent, or (b) a tyrosinase competing agent, or (c) a melanin reducing agent, or (d) combinations thereof, and
(ii) At least one antioxidant composition, and
(iii) At least one sunscreen composition.

4. A composition according to claim 1 wherein,
(i) From about 0.0001% to about 40% of at least one (a) a tyrosinase inhibiting agent, or (b) a tyrosinase competing agent, or (c) a melanin reducing agent, or (d) combinations thereof, and
(ii) From about 0.0001% to about 10% of at least one antioxidant composition, and,
(iii) From about 1% to about 99% of a cosmetically or pharmaceutically suitable carrier composition.

5. A composition according to claim 1 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

6. A composition according to claim 1 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

7. The compositions according to claim 1 wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.

8. A composition according to claim 2 wherein,
(v) From about 0.0001% to about 40% of at least one (a) a tyrosinase inhibiting agent, or (b) a tyrosinase competing agent, or (c) a melanin reducing agent, or (d) combinations thereof, and
(vi) From about 0.0001% to about 10% of at least one antioxidant composition, and,
(vii) From about 0.0001% to about 40% of at least one UV inhibitor, and
(viii) From about 1% to about 99% of a cosmetically or pharmaceutically suitable carrier composition.

9. A composition according to claim 2 wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

10. A composition according to claim 2 wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

11. The compositions according to claim 2 wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.

12. A composition according to claim 3 wherein,
(i) From about 0.0001% to about 40% of at least one (a) a tyrosinase inhibiting agent, or (b) a tyrosinase competing agent, or (c) a melanin reducing agent, or (d) combinations thereof, and
(ii) From about 0.0001% to about 10% of at least one antioxidant composition, and,
(iii) From about 1.0% to about 99.0% of at least one sunscreen composition.

13. A composition according to claim 3 wherein sunscreen composition can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

14. The compositions according to claim 3 wherein sunscreen composition can be traditional water and oil emulsions, suspensions, colloids, microemulsions, clear solutions, suspensions of nanoparticles, emulsions of nanoparticles, powders, or anhydrous compositions.
